(19) United States
(12) Patent Application Publication
(10) Pub. No.: US 2014/0004523 A1
(43) Pub. Date: Jan. 2, 2014

(54) SYSTEMS, METHODS, AND A KIT FOR DETERMINING THE PRESENCE OF FLUIDS ASSOCIATED WITH A HYDROCARBON RESERVOIR IN HYDRAULIC FRACTURING

(71) Applicants: Justine S. Chow, Durham, NC (US); Jake Garber Rudolph, JR., Durham, NC (US)

(72) Inventors: Justine S. Chow, Durham, NC (US); Jake Garber Rudolph, JR., Durham, NC (US)

(21) Appl. No.: 13/684,679

(22) Filed: Nov. 26, 2012

Related U.S. Application Data
(60) Provisional application No. 61/666,843, filed on Jun. 30, 2012.

(51) Int. Cl. C12Q 1/68

(52) U.S. Cl. CPC C12Q 1/6853
USPC 435/6.12; 536/23.1; 204/456

(57) ABSTRACT

Systems and methods and a kit are provided for using a DNA tracer for the detection of hydraulic fracturing fluid, and further including a method of creating well-specific tracers.

### BACKGROUND OF THE INVENTION

1. Field of the Invention

The present invention relates to hydraulic fracturing, and more particularly, to hydraulic fracturing in relation to tracers and ground water or drinking water monitoring.

2. Description of the Prior Art

Generally, it is known in the relevant art to provide DNA tracers used in hydraulic fracturing to detect possible groundwater contamination.

By way of background for the present invention, hydraulic fracturing has been around for many years, and is used for releasing natural gas, petroleum, shale gas, and other such substances from the ground. Hydraulic fracturing, which uses fluids injected underground into wells in order to stimulate natural gas or oil production, particularly in areas where there is not enough conductivity for production to be economically feasible using conventional drilling methods alone. Normally, these fluids consist of water or other solvent mixed with sand and various chemical mixtures that vary between different drilling companies. This mixture has been suspected of contaminating ground water and drinking water supplies. Although there have been attempts to resolve the sources of contamination for these cases, a great deal of uncertainty remains due to the inadequacy or lack of baseline water testing and the presence of other sources of possible contamination.

Although this process is a good method for extracting such substances, it is controversial. The controversy revolves around potential contamination of groundwater, as well as contamination of air and various other health risks. Currently, many of the methods and compounds used in the hydraulic fracturing process fall under trade secret protection, and thus there are minimal tests available for assessing potential contamination.

Generally, it is known that drilling companies utilize tracer services, but currently only for the purposes of fracture diagnostics during the exploratory phase of drilling, not during the hydraulic fracturing phase for the purpose of determining water contamination.

Tracer technology has many functions, ranging from testing backflow from hydraulic fracturing to output of fluids. There is currently a lack of patented ideas that would embody DNA tracer technology stabile enough to withstand shearing forces to test groundwater without being toxic specifically aimed at hydraulic fracturing. Most hydraulic fracturing related patents and patent applications deal with oil recovery and the measure of backflow.

Examples of relevant documents in the field include:

U.S. Pat. No. 8,143,388 by Soderbund, et al., for “Method and test kit for quantitative determination of polynucleotides in a mixture,” filed Apr. 18, 2008, and issued Mar. 27, 2012, describes a method and test kit for quantitative determination of the amounts of or relative proportions of polynucleotides in a mixture. The test kit includes one or more probe pools, each pool comprising: more than one soluble tracer-tagged polynucleotide probe, wherein each single tracer-tagged polynucleotide probe is complementary to an individual target polynucleotide sequence in the sample; one or more vessels, wherein each pool of polynucleotide probes is placed in its own vessel, wherein when multiple vessels are provided, they may be separate or joined together; and an apparatus for separating said tracer-tagged polynucleotide probes. Also, the polynucleotide probes are selected from the group consisting of DNA fragments, synthetic peptidic nucleic acids (PNAs), and locked nucleic acids (LNAs). The test kit also used in a method for quantitatively determining the amounts of multiple analyte polynucleotides present in a cell or tissue sample.

US Patent Application Pub. No. 20100307745 by Lafitte, et al., for “Use of encapsulated tracers,” filed Jun. 3, 2009 and published Dec. 9, 2010, describes a process in using distinguishable sets of tracer particles in subterranean reservoirs used with hydraulic fracturing by placing the distinguishable sets of particles in different locations within the veins and/or different veins extending from a single area, wherein the particles within each set of tracers contain unique substances distinguishing one set of tracers from another. Also, the tracer particles are encapsulated, the tracers are of different particle size and weight, the size ranging from 10 microns to 100 microns, and the tracers are released into the hydraulic fractures via a wellbore.

U.S. Pat. No. 6,645,769 by Tayebi, et al., for “Reservoir monitoring,” filed Nov. 29, 2000 and issued Nov. 11, 2003, describes a method for monitoring hydrocarbon and water production from areas and zones to detect changes in pH, composition, salinity, and microorganisms, using tracers that are zone/area specific and are unique to that area. The method is taught for application in a local alarm system for water breakthrough or for improved oil and gas recovery (IOR) in horizontal production and injection wells. Also disclosed is a monitoring system used for specific areas/zones for detection of different phenomena, including injecting specific tracers unique to that area or zone, wherein the tracers are immobilized and are chemically intelligent, released when they come in contact with oil or gas, and comprised of DNA, fluorescence, microorganisms, phosphorescent, or magnetic particles or fluids. The method focuses on the detection of the microorganism in zones or areas where they respond to specific stimulants, such as by fluorescence and phosphorescent, such detection being measured downstream.

U.S. Pat. No. 7, 560,690 by Stray, et al., for “System for delivery of a tracer in fluid transport systems and use thereof,” filed Jun. 30, 2005 and issued Jul. 14, 2009, describes a specific tracer delivery system composed of melamine formaldehyde resin (MFR) doped with various tracer materials, wherein the MFR is used to slowly release tracer compounds into a liquid system. The MFR can be doped with different types of tracers, thereby allowing placement of different tracers at several different positions upstream, and production from the various labeled zones can be verified through the analysis of one sample downstream. The MFR, combined with tracer materials which can be both radioactive and non-radioactive, is measured upstream and then later downstream. Radioactive tracers can be filled with fillers, plasticisers, stabilizers, and colorants, whereas a non-radioactive tracer may include naphthalenesulphonic acid, amino naphthalenesulphonic acid, fluorinated benzoic acid or salts thereof, and may further be comprised of fillers, plasticizers, stabilizers and/or colorants. The polymer tracers are active in the system both upstream and downstream for up to 1 year.

U.S. Pat. No. 7, 339,160 by Raghuraman, et al., for “Apparatus and method for analysing downhole water chemistry,” filed Nov. 19, 2003 and issued Mar. 4, 2008, describes an apparatus for analyzing water chemistry that is used underground and provides a colouring agent to water samples that indicate the water sample chemistry. Furthermore, the apparatus has an injector which is introduced to the flowline, the flowline then being injected with a color and later mixed with a double helix mixture to determine water chemistry by a colorimetric analyzer. Also disclosed is a monitoring system for “downhole” water contamination by adding a tracer to the contaminated water and further adding a colouring agent, as mentioned above, to determine water chemistry.

No prior art is known to provide tracers for detecting contamination in water by hydraulic fracturing fluids, as with the present invention. Thus, there remains a need in the art to provide methods and systems for detecting contamination in water from hydraulic fracturing fluids.

### SUMMARY OF THE INVENTION

It is a primary object of the present invention to provide methods and systems for using a tracer for the detection of hydraulic fracturing fluid in suspected areas of hydraulic fracturing fluid contamination, particularly contamination of water, using biopolymers.

A further object of the present invention is to provide methods and systems for using a tracer for the detection of hydraulic fracturing fluid, wherein tracer variation such that contamination deriving from individual wells can be distinguished from each other.

Another object of the present invention is to provide a method of adding biopolymers to hydraulic fracturing fluid for detection without increasing toxicity or radioactivity of the hydraulic fracturing fluid.

Still another object of the present invention is to provide a tracer that is able to endure hydraulic fracturing fluid conditions, which includes durability against high salinity, high temperatures (above about 70° C.), high pressures, and shearing forces.

These and other objects and aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A is a diagram of one embodiment of the invention, illustrating depictions of a secondary structure of the tracer using mfold software.

FIG. 1B is a diagram of the energy dot plot of the structure, shown in FIG. 1A, illustrating the free energy of the tracer structure.

FIG. 1C is another software-generated diagram of one embodiment of the invention, illustrating DNA tracer structure.

### DETAILED DESCRIPTION

Referring now to the drawings in general, the illustrations are for the purpose of describing a preferred embodiment of the invention and are not intended to limit the invention thereto.

The present invention provides systems, methods, and a kit for use with hydraulic fracturing in relation to tracers and ground water or drinking water monitoring. While the prior art provides for improvements on DNA tracers used in testing and measuring liquids, none are known to be applicable to measure the safety level of drinking water whilst not contaminating the water.

The present invention provides systems and methods and a kit for using a tracer for the detection of hydraulic fracturing fluid, including a method of creating well-specific tracers, and further a method of applying and interpreting water samples that may contain the tracer after the tracer's application to a hydraulic fracturing fluid.

The tracer consists of nucleotide strands, which are biopolymers that consist of a sugar, phosphate group and nucelobases or nucleobase analogues. In preferred embodiments, nucleotides are used instead of nucleotide analogues. The nucleobase is a nitrogen-based molecule that, in DNA, forms hydrogen-bonded pairs that form the bridge between the two nucleotide strands. Nucleobase analogues include non-nitrogen bases that may not attach to each other, but are still able to form sequences along the length of the nucleotide strands. The nucleotide or nucleotide analogue also includes a five-carbon sugar, either ribose or 2-deoxyribose, and a phosphate group, PO43-. These resulting nucleotide strands contain sequences that can be customized as a unique tag for each individual tracer, designed for a specific well. The resulting strands are also able to form three dimensional (3-D) structures through specific hydrogen bonding formed from the sequences, increasing the compactness.

The 3-D structures, which can include hairpin structures, loops, or scaffolding configurations, decrease exposure to high shear and increases resistance to temperature or chemical degradation. Preferably, a single hairpin structure is provided that includes a base step loop section that confers durability and resilience, and a pair of dangling ends that are used for identification. The length of the strand and the distribution of specific types of nucleobases also increase the strand's strength. According to methods of the present invention, a tracer is mixed with water and added to hydraulic fracturing fluids before they are injected into hydraulic fracturing wells. Material from a source suspected to be contaminated by hydraulic fracturing fluids is later analyzed for the tracer. Individual tracer sequences are matched to individual wells, identifying the exact well that is the source of contamination, thus providing well-specific tracers for identifying contamination by hydraulic fracturing fluids.

The present invention provides a method for determining the presence of fluids associated with a hydrocarbon reservoir used in hydraulic fracturing, including the steps of: synthesizing a tracer comprising a nucleotide or nucleotide analogue strand, wherein the tracer is capable of surviving hydraulic fracturing conditions; matching the sequence of the tracer with a specific well; diluting the tracer with water and inserting the mixture thus obtained into the hydraulic fracturing fluid; and analyzing environmental samples, such as groundwater, through methods such as polymerase chain reaction (PCR) or array-based electrical detection to determine whether the tracer is present.

Also, the tracer consists of nucleotide or nucleotide analogue sequences that inherently allow for variation diverse enough for each drilling well to have its own tracer within a drilling area. Preferably, the tracer may be diluted with water and added directly to the hydraulic fracturing fluid without needing any other additional materials more toxic than water. The tracer itself consists of material that is biologically inert and does not pose significant harm to biological systems; thus, the tracer is not more toxic to the environment than the hydraulic fracturing fluid to which it is being added. Also, the tracer is able to withstand the high salinity, acidic pH, and high metal ion content that is typically found in the surrounding fluid. Furthermore, the tracer is long enough and therefore durable enough to enable a detection temperature of above about 70° C. In another embodiment the tracer is long enough and durable enough to enable a detection temperature between about 70° C. and 100° C. Also, preferably, the tracer is able to form 3-D configurations that enable it to withstand shearing forces capable of pulling apart long unfolded sugar and phosphate chains.

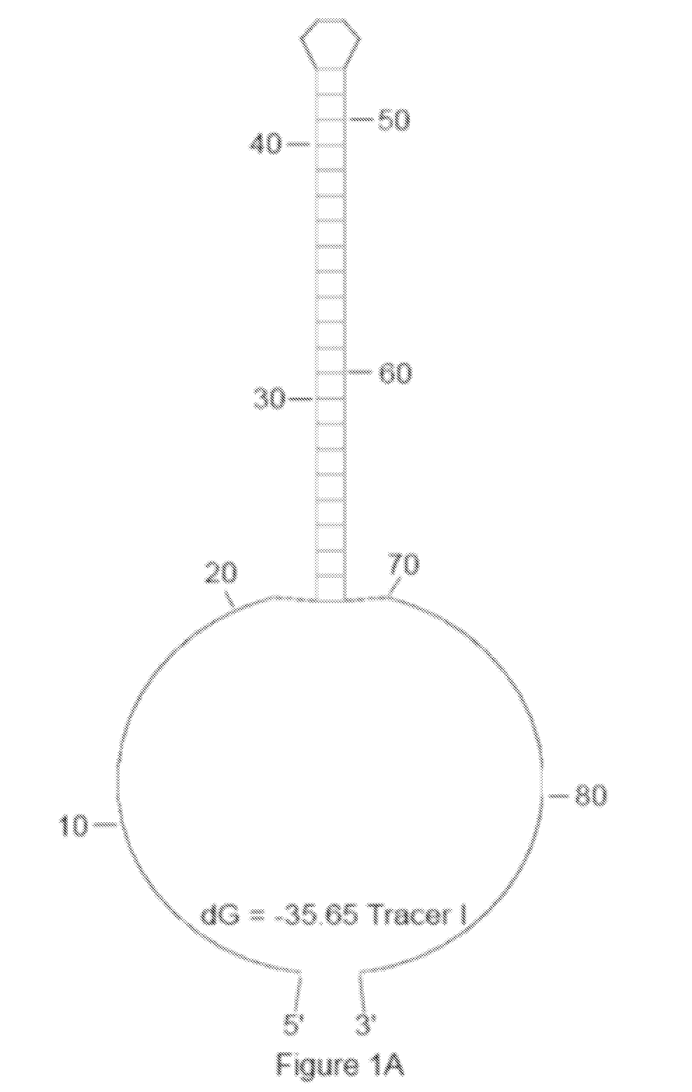

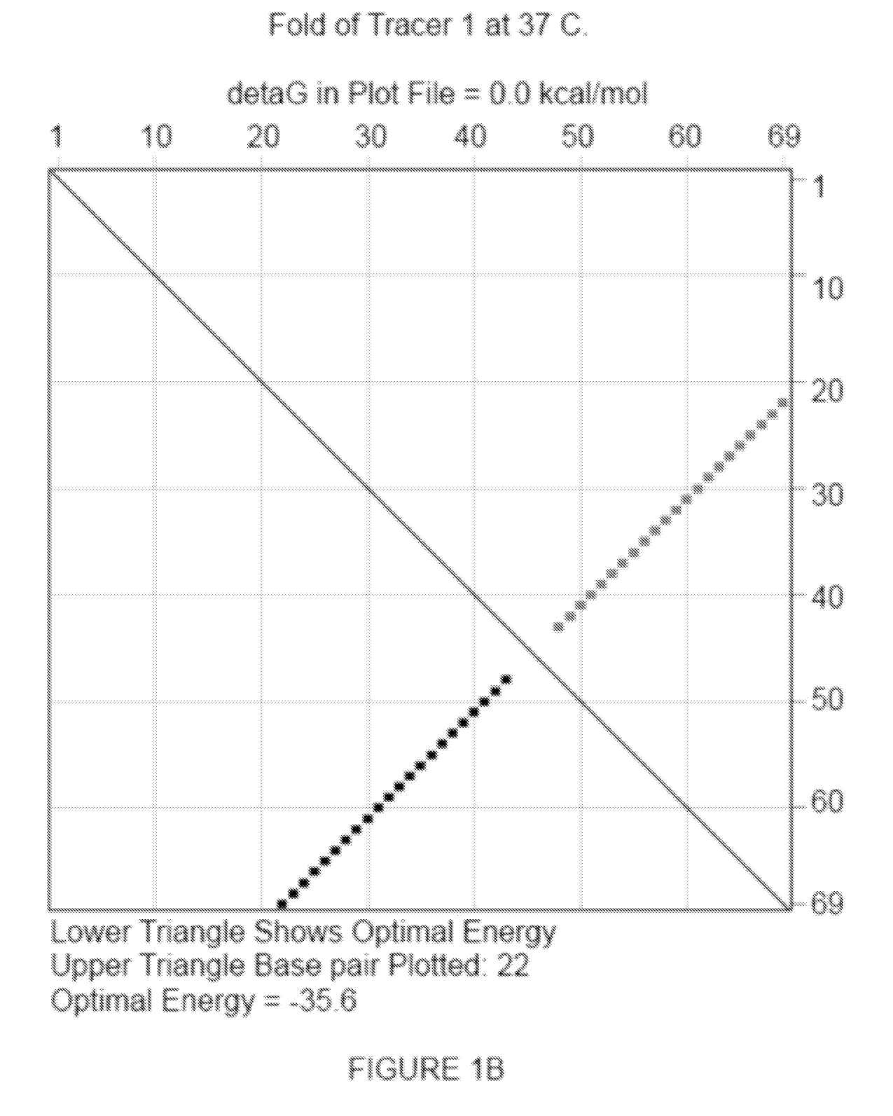

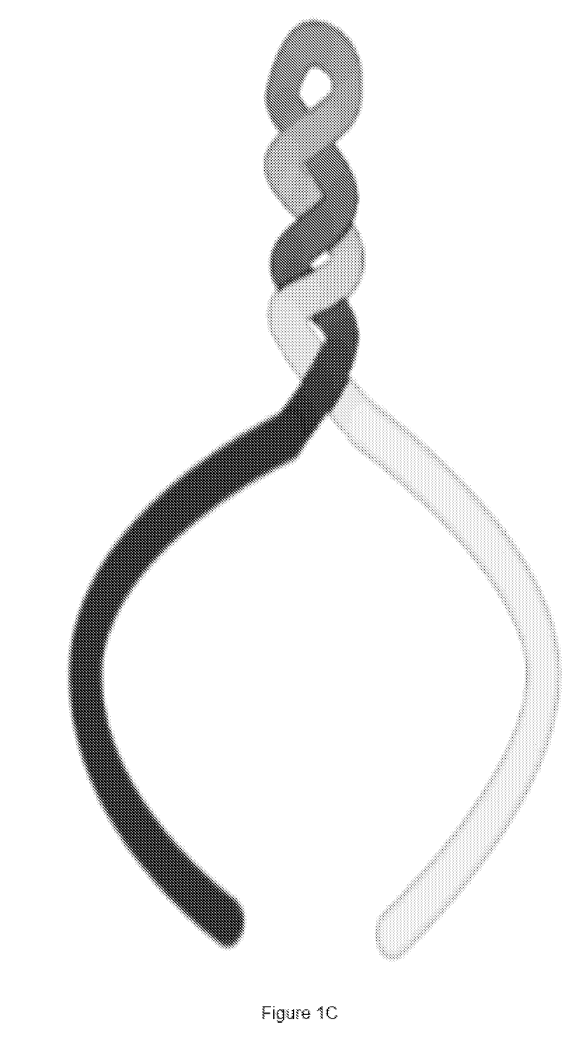

FIG. 1A is a diagram of one embodiment of the invention, illustrating depictions of a secondary structure of the tracer using mfold software. FIG. 1B is a diagram of the embodiment of the invention shown in FIG. 1A, illustrating the free energy (ΔG) of the tracer structure. FIG. 1C is an illustrated three-dimensional diagram of one embodiment of the invention, showing the DNA tracer structure. In particular, as illustrated, the present invention uniquely provides DNA tracer methods, systems, and a kit used for detecting contamination of water or other fluid by hydraulic fracturing fluids, wherein the tracers include at least 60% G-C base pair content. Also, the tracer is characterized by an extremely strong “loop” at the middle of the sequence and a double-stranded stem that confers durability, while making the structure compact enough to withstand shearing forces. The tracer has unique identifier dangling ends that can be switched out for different wells for providing well-specific tracers. Notably, there is a hairpin structure of the tracer that unfolds at close to 100° C.; also, it does not degrade at higher temperatures.

In methods for molecule specification according to the present invention at least one DNA tracer is provided, wherein the tracer consists of a DNA sequence, a nucleic acid. Such a sequence is artificially synthesized and not found naturally according to National Institute of Health Basic Local Alignment Search Tool (BLAST). The DNA tracer is a single strand folded approximately in half, such that part of it is double stranded with another part of the strand, with a loop at its fold, forming a “hairpin” structure, and dangling ends that do not pair with the other ends and are free-floating single-stranded DNA, as illustrated in the FIGS. 1A, 1B, 1C.

The method steps further include adding the tracer to hydraulic fracturing fluid during the regular mixing process for hydraulic fracturing. If there is a continuous stream from mixing to injection the tracer is mixed into the fluid near the beginning of injection. Flowback or produced water is provided for sampling and for confirmation that the tracer is present in the water.

In methods of using the tracers, systems and kits for testing groundwater according to the present invention, water samples are provided. These water samples are cleaned with an ethanol rinse for PCR inhibitor removal, sequences are amplified by polymerase chain reaction (PCR), and results are detected using a detection method, e.g., gel electrophoresis. Two sets of testing are provided: a first set of testing to detect the presence or absence of the DNA tracer(s) according to the present invention, as described hereinabove, either through a mix of multiple primers in PCR or through an universal tracer that interacts with the DNA tracer(s); and a second set of testing that is performed only in the case of a positive result or an uncertain result from the first set of testing. The second set of testing identifies which set of dangling ends were used with the DNA tracer(s) detected. The step of identifying the set of dangling ends includes isolating testing of individual pairs of primers in PCR and narrowing down or reducing the results to match a specific well (i.e., detecting well-specific tracers).

Certain modifications and improvements will occur to those skilled in the art upon a reading of the foregoing description. By way of example and not limitation, the methods, systems, and kit according to the present invention, while described for application to detection of contamination by hydraulic fracturing fluids, may be applied for detection and tracking of water rights, tracing groundwater or surface water systems for scientific analysis and tracing, for example the study of geology for environmental remediation, tracing chemicals, waste or other fluids for the purposes of accountability in other fields, carbon sequestration and/or detecting leakage of liquefied carbon dioxide, and/or tracing fuels. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

What is claimed is:

1. A method for determining contamination of fluids comprising the steps of:
providing samples of a fluid;
cleaning the samples;
conducting a first set of testing to detect the presence or absence of a DNA tracer,
wherein the DNA tracer includes a hairpin configuration and a set of dangling ends, thereby providing testing steps for determining contamination of fluids by detecting the presence of the DNA tracer in the fluid tested.

2. The method of claim 1, wherein the step of detecting the presence of the DNA tracer further includes using a mix of multiple primers in polymerase chain reaction (PCR).

3. The method of claim 1, wherein the step of detecting the presence of the DNA tracer further includes using an universal tracer that interacts with the DNA tracer.

4. The method of claim 1, further including the step of conducting a second set of testing that is performed in the case of a positive result from the first set of testing.

5. The method of claim 1, further including the step of conducting a second set of testing that is performed in the case of an uncertain result from the first set of testing.

6. The method of claim 4, further including a step of identifying a set of dangling ends that further includes isolating testing of individual pairs of primers in PCR.

7. The method of claim 6, further including the step of reducing the results of the second set of testing to match a specific fluid source.

8. The method of claim 1, wherein the samples are cleaned with an ethanol rinse or polymerase chain reaction (PCR) inhibitor removal for sequence amplification.

9. The method of claim 1, wherein the detection method includes gel electrophoresis.

10. A tracer for determining contamination of fluids, the tracer comprising a DNA structure including a hairpin and dangling ends, wherein the structure is durable, and wherein the structure is provided for detection of source.

11. The tracer of claim 10, wherein the hairpin includes a single strand of DNA folded approximately in half, such that part of the single strand is double stranded with another part of the single strand, with a loop at its fold, the loop forming the hairpin.

12. The tracer of claim 10, wherein the dangling ends do not pair with other ends of a single strand of the DNA structure.

13. The tracer of claim 10, wherein the dangling ends have a free-floating single-stranded DNA structure.

14. A system for determining contamination of fluids comprising at least one tracer, the at least one tracer comprising a DNA structure including a hairpin and dangling ends, wherein the structure is durable, and wherein the structure is provided for detection of source.

15. The system of claim 14, wherein the hairpin includes a single strand of DNA folded approximately in half, such that part of the single strand is double stranded with another part of the single strand, with a loop at its fold, the loop forming the hairpin.

16. The system of claim 14, wherein the dangling ends do not pair with other ends of a single strand of the DNA structure.

17. The system of claim 14, wherein the dangling ends have a free-floating single-stranded DNA structure.

18. The system of claim 14, wherein the at least one tracer further includes one fluid-source specific tracer.

19. The system of claim 18, wherein the at least one fluid-source specific tracer is associated with a groundwater source.
